(19) United States
(12) Patent Application Publication
(10) Pub. No.: US 2011/0245637 A1
(43) Pub. Date: Oct. 6, 2011

(54) AMBIENT LIGHT USE IN PHYSIOLOGICAL SENSORS

(75) Inventor: Edward M. McKenna, Boulder, CO (US)

(73) Assignee: Nellcor Puritan Bennett LLC, Boulder, CO (US)

(21) Appl. No.: 12/751,801

(22) Filed: Mar. 31, 2010

(51) Int. Cl. A61B 5/1455; G01J 3/42

(52) U.S. Cl. 600/310; 356/319

(57) ABSTRACT

The present disclosure describes the harvesting and use of ambient light in spectrophotometric systems so as to increase the energy efficiency of the systems. In one embodiment, the collected ambient light is filtered and/or converted into discrete wavelengths of light that can then be used in spectrophotometric applications. In one embodiment, the emitted light can then be collected and analyzed to derive various physiological parameters. In certain embodiments, the ambient light may be used in place of light that is electrically generated.

### BACKGROUND

The present disclosure relates generally to medical diagnostic sensors and, more particularly, to energy efficient spectrophotometric sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor and sense certain physiological characteristics of their patients. Accordingly, a wide variety of devices have been developed for monitoring and sensing many such physiological characteristics. One category of monitoring and sensing devices includes spectrophotometric monitors and sensors. This category of device studies the electromagnetic spectra (e.g., wavelengths of light) and can monitor a suite of physiological parameters. Such devices provide doctors and other healthcare personnel with the information they need to provide the best possible healthcare for their patients. As a result, such monitoring and sensing devices have become an indispensable part of modern medicine.

One technique for monitoring certain physiological characteristics of a patient using spectrophotometric devices is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin (SpO₂) in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed and/or scattered. More specifically, the light passed through the tissue is typically selected to be of one or more wavelengths that may be absorbed and/or scattered by the blood in an amount correlative to the amount of a blood constituent present in the blood. The amount of light absorbed and/or scattered may then be used to estimate the amount of blood constituent in the tissue using various algorithms. This determination may be performed in a monitor coupled to the sensor that receives the necessary data for the blood constituent calculation.

Conventional pulse oximeter sensors are typically connected to a monitor via a cable. The cable provides the sensor with power and acts as a conduit for the transmission of signals between the sensor and the monitor. However, the cable also acts to tether the patient to the monitor, preventing unencumbered motion by the patient. As a result, such cable-based systems may not be suitable for ambulatory patients or for applications that require remote monitoring in non-clinical environments. Accordingly, various systems have been proposed which include a patient sensing device connected to a local monitor by way of a wireless link. Such wireless devices, however, may only be able to operate for a limited time due to constraints on the amount of available power (e.g., battery power) provided on the wireless device.

### BRIEF DESCRIPTION OF THE DRAWINGS

Advantages of the disclosed techniques may become apparent upon reading the following detailed description and upon reference to the drawings in which:

FIG. 1 illustrates components of a spectrophotometric system, in accordance with one embodiment of the present disclosure;

FIG. 2 depicts a block diagram of a spectrophotometric wireless sensor and monitor, in accordance with one embodiment of the present disclosure;

FIG. 3 illustrates an embodiment of the wireless sensor of FIG. 2 collecting ambient light through a lens-like structure, in accordance with one embodiment of the present disclosure;

FIG. 4 illustrates an embodiment of the wireless sensor of FIG. 2 collecting light through a fiber optic woven fiber, in accordance with one embodiment of the present disclosure;

FIG. 5 depicts a graph of the intensity of ambient fluorescent light at different wavelengths showing the filtering of a portion of the ambient fluorescent light, in accordance with one embodiment of the present disclosure;

FIG. 6 depicts a graph of the intensity of ambient fluorescent light at different wavelengths showing the conversion of a portion of the ambient fluorescent light, in accordance with one embodiment of the present disclosure;

FIG. 7 depicts a graph of the intensity of ambient fluorescent light at different wavelengths showing the filtering and the conversion of a portion of the ambient fluorescent light, in accordance with one embodiment of the present disclosure;

FIG. 8 illustrates an embodiment of the wireless sensor of FIG. 4 wherein the ambient light collector has been provided as part of a garment, in accordance with one embodiment of the present disclosure;

FIG. 9 illustrates an embodiment of the wireless sensor of FIG. 4 wherein the ambient light collector has been provided as part of a bandage, in accordance with one embodiment of the present disclosure;

FIG. 10 depicts an embodiment of the wireless sensor of FIG. 4 wherein the ambient light collector comprises a lens-like structure, in accordance with one embodiment of the present disclosure; and

FIG. 11 depicts an embodiment of the wireless sensor of FIG. 4 wherein the ambient light collector comprises a fiber optic woven or knit garment, in accordance with one embodiment of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure relates various techniques by which a spectrophotometric sensor may utilize ambient light in the sensor environment. By utilizing ambient light, a spectrophotometric sensor may be able to avoid generating light as part of its operation or may reduce the amount of light generated, thereby conserving the energy typically associated with light generation. In this manner, a wireless sensor may be able to reduce energy consumption when operating wirelessly.

Present embodiments may apply to a variety of wireless spectrophotometric sensors, such as pulse oximetery sensors. Moreover, as disclosed herein, the data of interest that may be observed using a wireless spectrophotometric sensor may vary depending on the capabilities of each device. For example, a pulse oximetry sensor may transmit data of interest that includes pulse rate, blood oxygen saturation, and/or total hemoglobin, and so forth. Because the embodiments presently disclosed may reduce the amount of light produced by the sensor electronics, the wireless sensors may expend less power and, accordingly, may have a longer battery life and/or may employ smaller or less expensive batteries.

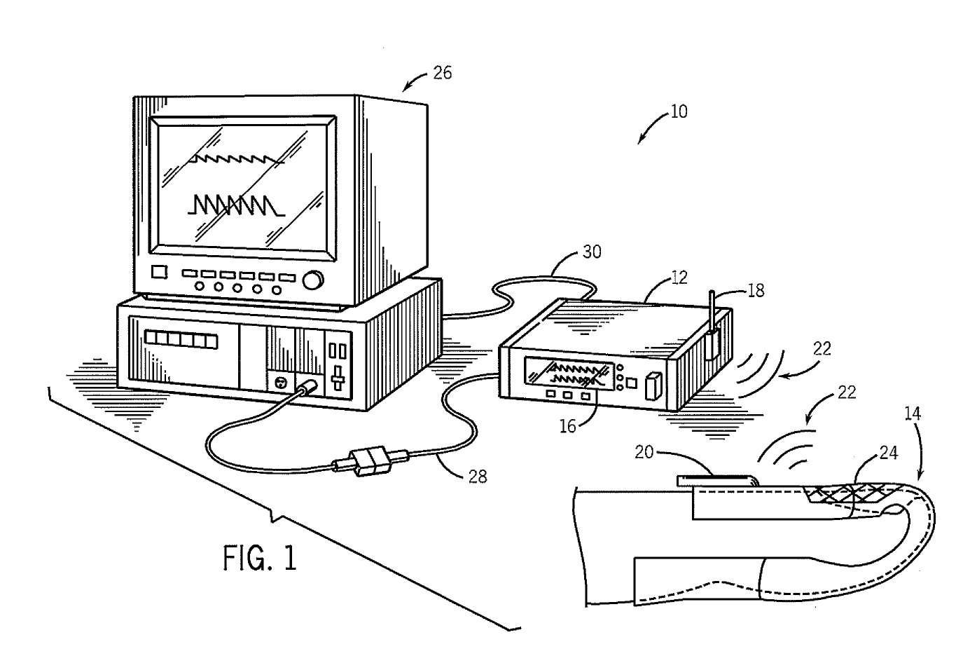

With the foregoing in mind, FIG. 1 depicts an embodiment of a wireless monitoring system 10 that may efficiently collect and utilize ambient light, thereby conserving power. The system 10 may include a patient monitor 12 that communicates wirelessly with a wireless sensor 14. The patient monitor 12 may include a display 16, a wireless module 18 for transmitting and receiving wireless data, a memory, a processor, and various monitoring and control features. Based on diagnostic sensor data received from the wireless sensor 14, the patient monitor 12 may display patient diagnostic measurements and perform various additional algorithms. For example, when the system 10 is configured for pulse oximetry, the patient monitor may perform blood oxygen saturation calculations, pulse measurements, and other measurements based on the received wireless sensor data. Furthermore, to upgrade conventional operations provided by the monitor 12 to provide additional functions, monitor 12 may be coupled to a multi-parameter patient monitor 26 via a cable 28 connected to a sensor input port or via a cable 30 connected to a digital communication port, for example.

In one embodiment of the system 10, the wireless sensor 14 may be a pulse oximeter sensor. However, the sensor 14 may also be other types of spectrophotometric sensors, such as sensors capable of obtaining any of a variety of medical spectrophotometric diagnostic measurements, such as transvascular fluid exchange volumes, bilirubin levels, non-invasive blood pressures (NIBP), tissue hydration, and so forth.

Like the patient monitor 12, the sensor 14 may include a wireless module 20. The wireless module 20 of the sensor 14 may establish wireless communication 22 with the wireless module 18 of the patient monitor 12 using any suitable protocol. By way of example, the wireless module 20 may be capable of communicating using the IEEE 802.15.4 standard, and may communicate, for example, using ZigBee, WirelessHART, or MiWi protocols. Additionally or alternatively, the wireless module 20 may be capable of communicating using the Bluetooth standard or one or more of the IEEE 802.11 standards.

As described further below, in one embodiment the sensor 14 may collect ambient light for use via an ambient light collector 24, such as a light collecting portion of the sensor 14 itself or a light collector in optical communication with the sensor 14. The collected ambient light may be used to replace or to complement light emitted by electrical components within the sensor 14, such as by an LED. The sensor 14 may also utilize other energy harvesting techniques such as kinetic energy transducers, solar power, thermoelectrics, ambient RF collection, and others to extend battery life and improve power efficiency.

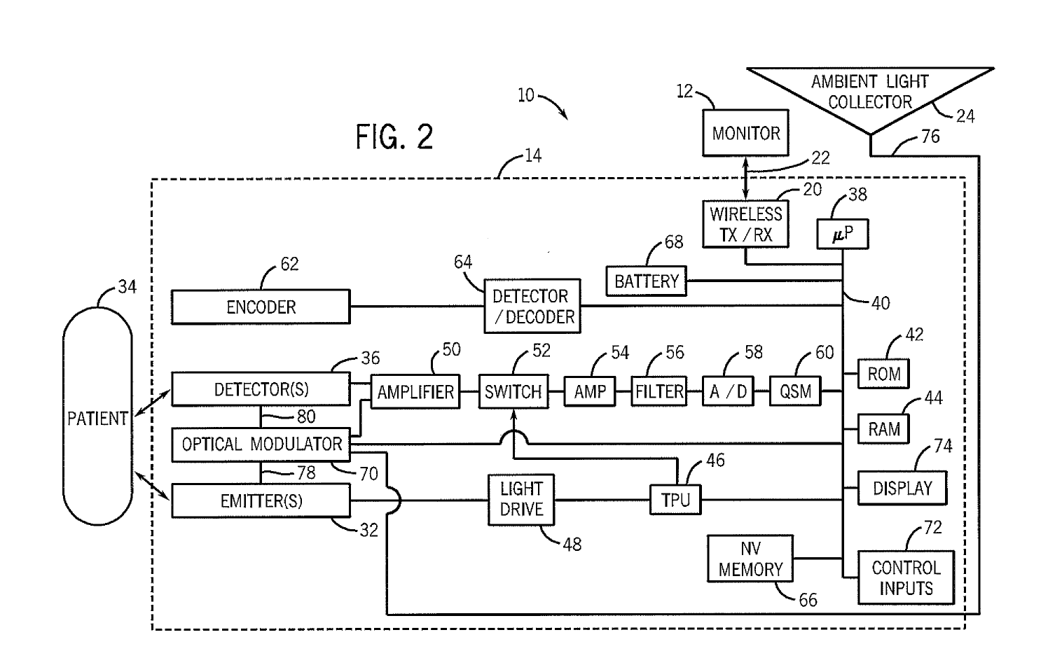

FIG. 2 is a block diagram of an embodiment of the wireless monitoring system 10 that may be configured to implement the techniques described herein. By way of example, embodiments of the system 10 may be implemented with any suitable sensor and patient monitor, such as those available from Nellcor Puritan Bennett LLC. The system 10 may include the patient monitor 12 and the sensor 14, which may be configured to obtain, for example, a plethysmographic signal from patient tissue at certain predetermined wavelengths. The sensor 14 may be communicatively connected to the patient monitor 12 via wireless communication 22. When the system 10 is operating, light from the emitter(s) 32 may pass into the patient tissue 34, may be absorbed and/or scattered by the tissue, and may be detected by the detector(s) 36. As discussed herein, the emitted light may be derived, partially or completely, from ambient light collected in the vicinity of the system 10 and/or patient.

In one embodiment, the sensor 14 is capable of driving the data collection process and/or of processing and/or storing the acquired data. For example, the sensor 14 may include a microprocessor 38 connected to an internal bus 40. Also connected to the bus 40 may be a ROM memory 42 and a RAM memory 44. A time processing unit (TPU) 46 may provide timing control signals to light drive circuitry 48 which may control when light is emitted, and if multiple light wavelengths are emitted, the multiplexed timing for the different wavelengths. The TPU 46 may also control the gating-in of signals from the detector(s) 36 and optical modulator 70 through an amplifier 50 and a switching circuit 52. These signals may be sampled at the proper time, depending upon the timing associated with different wavelength emissions. In certain embodiments, the received signal from the detector 36 and/or optical modulator 70 may be passed through an amplifier 54, a low pass filter 56, and/or an analog-to-digital converter 58.

The digital data may then be stored in a queued serial module (QSM) 60, for later downloading to the RAM 44 as the QSM 60 fills up. In one embodiment, there may be separate parallel paths of separate amplifier, filter and A/D converters for respective light wavelengths or spectra received. This raw digital diagnostic data may be further sampled by the circuitry of the sensor 14 into specific diagnostic data of interest, such as pulse rate, blood oxygen saturation, and so forth. The diagnostic data of interest may take up significantly less storage space than the raw diagnostic data. For example, a raw 16-bit digital stream of photoplethysmographic data sampled at 100 Hz may be further sampled to obtain an instantaneous pulse rate at a given time, which may take up only approximately 8 bits.

In an embodiment, the sensor 14 may also contain an encoder 62 that provides signals indicative of the wavelength of one or more light sources of the emitter 32, which may allow for selection of appropriate calibration coefficients for calculating a physiological parameter such as blood oxygen saturation. The encoder 62 may, for instance, be a coded resistor, EEPROM or other coding devices (such as a capacitor, inductor, PROM, RFID, parallel resonant circuits, or a colorimetric indicator) that may provide a signal to the processor 38 related to the characteristics of the spectrophotometric sensor 14 that may allow the processor 38 to determine the appropriate calibration characteristics for the sensor 14. Further, the encoder 62 may include encryption coding that prevents a disposable part of the sensor 14 from being recognized by a processor 38 that is not able to decode the encryption. For example, a detector/decoder 64 may be required to translate information from the encoder 62 before it can be properly handled by the processor 38. In some embodiments, the encoder 62 and/or the detector/decoder 64 may not be present. Additionally or alternatively, the processor 38 may encode processed sensor data before transmission of the data to the patient monitor 12.

In various embodiments, based at least in part upon the value of the received signals corresponding to the light detected by detector 36, the microprocessor 38 may calculate a physiological parameter of interest using various algorithms. These algorithms may utilize coefficients, which may be empirically determined, corresponding to, for example, the wavelengths of light used. In one embodiment, these algorithms may be stored in the ROM 42. In a two-wavelength system, the particular set of coefficients chosen for any pair of wavelength spectra may be determined by the value indicated by the encoder 62 corresponding to a particular light source provided by the emitter(s) 32. For example, the first wavelength may be a wavelength that is highly sensitive to small quantities of deoxyhemoglobin in blood, and the second wavelength may be a complementary wavelength. For example, such wavelengths may be produced by orange, red, infrared, green, and/or yellow LEDs and/or by modulation, selection, and/or filtering of collected ambient light, as discussed herein. Different wavelengths may be selected based on instructions from the patient monitor 12 and/or based on preferences stored in the ROM 42 and/or a nonvolatile storage 66. The instructions from the patient monitor 12 may be transmitted wirelessly to the sensor 14 and may be selected at the patient monitor 12 by a switch on the patient monitor 12, a keyboard, or a port providing instructions from a remote host computer.

A set of control inputs 72 may allow the user to select different sensor modalities such as power utilization mode (e.g. standby power mode, low power mode, standard power mode, etc.), wireless update interval rate, transmission type (e.g., Bluetooth, 802.11, ZigBee, etc.), among others. A display 74 may be used to show the remaining battery life, the current power utilization mode, photoplethysmographic measurements, among others. Nonvolatile memory 66 may store caregiver preferences, patient information, or various parameters, discussed above, which may be used in the operation of the sensor 14. Software for performing the configuration of the sensor 14 and for carrying out the techniques described herein may also be stored on the nonvolatile memory 66, or may be stored on the ROM 42. The nonvolatile memory 66 and/or RAM 44 may also store historical values of various discrete medical diagnostic data points. By way of example, the nonvolatile memory 66 and/or RAM 44 may store values of instantaneous pulse rate for every second of the most recent five minutes. These stored values may be used as factors in determining the wireless data transfer rate, for example.

A battery 68 may supply the wireless sensor 14 with operating power. By way of example, the battery 68 may be a rechargeable battery, such as a lithium ion or lithium polymer battery, or may be a single-use battery such as an alkaline or lithium battery. Due to the techniques described herein to reduce battery consumption, the battery 68 may be of a much lower capacity, and accordingly much smaller and/or cheaper, than a battery needed to power a similar wireless sensor that does not employ the present techniques.

In one embodiment, the wireless sensor 14 may also include an optical modulator 70. The optical modulator 70 may receive incoming ambient light collected by an ambient light collector 24 using a waveguide 76 as the light transmission medium. Different embodiments of the ambient light collector 24 may be used, for example a lens-like structure, a fiber optic woven or knit garment, among others, may be used as ambient light collecting structures.

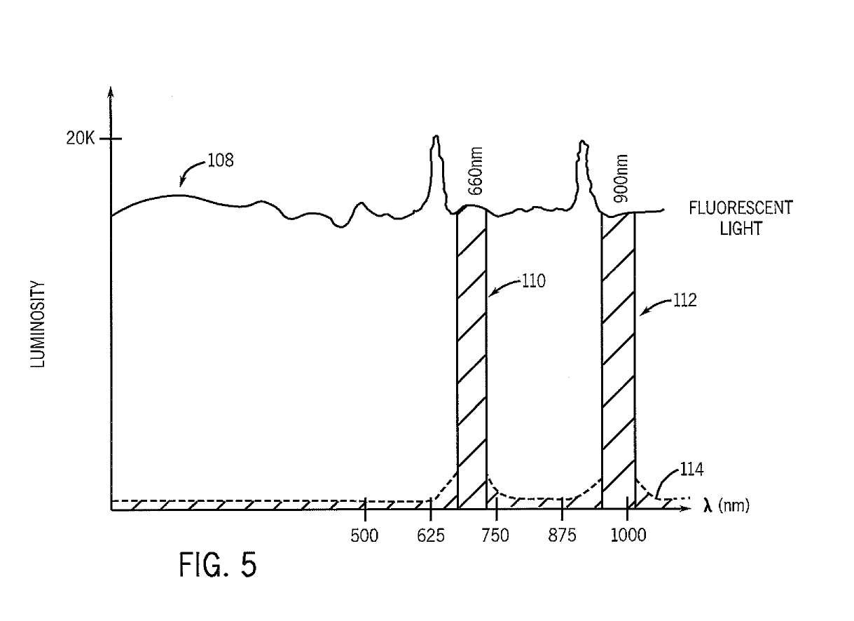

Ambient light entering or exiting the optical modulator may be filtered and/or converted, as described in more detail below with respect to FIGS. 5, 6, and 7, into light at discrete wavelengths which may then be emitted into a patient's tissue 34, such as by using a waveguide 78 as the transmission medium. The emitted light may be collected by detector(s) 36 positioned on an opposite surface of the tissue (i.e., a transmission mode) or on an adjacent surface of the tissue (i.e., a reflectance mode). In one embodiment the detector 36 may include structures, such as an optical fiber or fiber bundles, on the sensor 14 which may be used to collect light at the measurement site for transmission to a downstream photodetector component for conversion to an electrical signal. In other embodiments the photodetector component, e.g., one or more photodiodes, may be present at or near the measurement site such that the detected light is converted into an electrical signal effectively at the measurement site.

In one embodiment, light collected at the measurement site may be transmitted into the optical modulator 70 using a waveguide 80 as the light transmission medium. In one such an embodiment, the collected light may be filtered and modulated using the optical modulator 70 and subsequently converted into an electrical signal by an optical detector 36. In an embodiment, the optical modulator 70 may include a filtering element (e.g., a Bragg filter) to aid in reliably filtering the collected light. The electrical signal generated by the detector 36 may be processed through amplifier 50, switch 52, amplifier 54, low band filter 56, A/D converter 58, and QSM 60 as described earlier. For example, in one embodiment a white light source and/or unfiltered ambient light may be emitted into the tissue 34 and the detected light exiting the tissue may be provided to the optical modulator 70 to filter out and/or modulate wavelengths and signals that are not of interest, allowing detection and processing of only those wavelengths and signals that are of interest. Alternatively, to the extent that the light emitted into the tissue 34 consists only of those appropriately modulated wavelengths of interest (whether due to generation by wavelength specific LEDs or due to filtering and/or conversion of collected ambient light by the optical modulator 70), the detected light may bypass the optical modulator 70 and may instead proceed to amplification, A/D conversion, low band filtering, and so forth, as discussed above.

Microprocessor 38 may process the signal corresponding to detected light to calculate various physiological measurements of interest. Microprocessor 38 may also implement various optical controller modalities that allow the control of the optical modulator 70. The optical controller modalities may include the timing instructions that allow for the timed emission of a set of discrete wavelengths of light that may be converted and/or filtered by optical modulator 70 and emitted by emitter(s) 32.

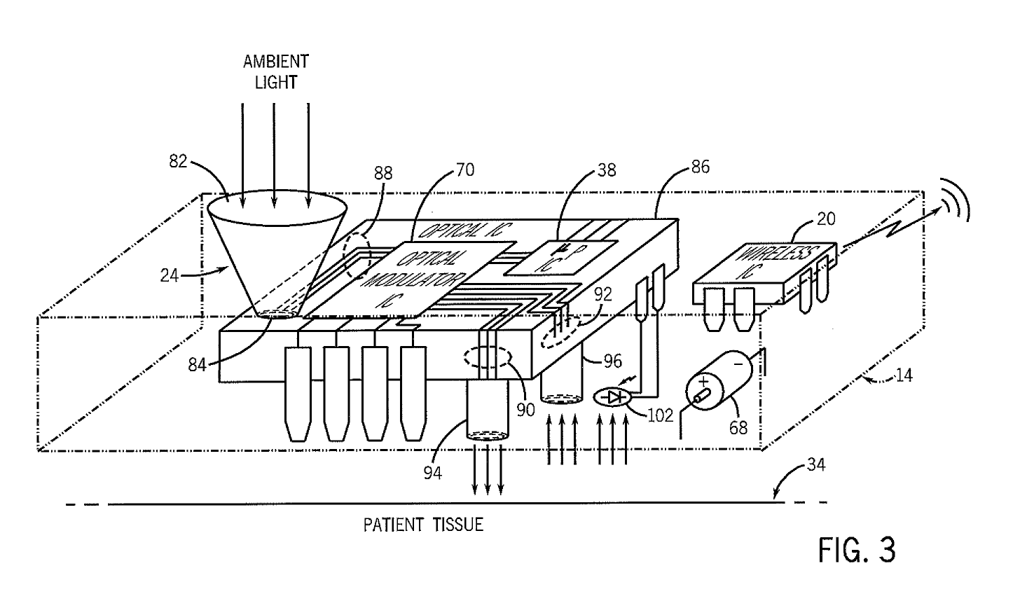

FIG. 3 illustrates certain optical and wireless components of one embodiment of a wireless sensor in which an ambient light collector 24 is provided as a lens or lens-like structure 82. The lens-like structure 82 may collect ambient light incident on the lens-like structure 82 and may focus the collected light into a light-receiving aperture 84 of an optical chip 86. On-chip waveguides 88 may transmit the collected ambient light from the light-receiving aperture 84 into the optical modulator 70. In one embodiment, the optical modulator 70 may filter the incoming ambient light and/or convert the incoming ambient light into discrete wavelengths of light that may be used in the operation of the sensor. For example, ambient light may be filtered and/or converted into specific, discrete wavelengths because of the utility of certain discrete wavelength ranges in areas of spectrophotometric monitoring such as pulse oximetry. For example, in one embodiment, the wavelengths of light near 620-700 nanometers (nm) and near 860-940 nm may be used to derive blood-oxygen saturation of hemoglobin (SpO₂) in arterial blood, the volume of individual blood pulsations supplying the tissue, and/or the rate of blood pulsations corresponding to each heartbeat of a patient. Other wavelength ranges may be used in addition or instead of the aforementioned wavelength ranges, depending on the spectrophotometric operations being performed.

FIG. 3 also depicts an emitter 94 and a light collector 96 that may be optically connected to the optical modulator 70. In one embodiment, one or both of the emitter 94 and light collector 96 may be passive, that is, the emitter 94 and the light collector 96 may be unlike traditional spectrophotometric emitters (e.g., LEDs) and detectors (e.g., photodiodes) in that the emitter 94 and/or the light collector 96 may not utilize electrical components to emit and/or to collect light. The emitter 94 and the light collector 96 may instead be connected to the optical modulator 70 via on-chip waveguides 90 and 92 respectively. In one embodiment, discrete wavelength ranges of light, for example light near 620-700 nm and near 860-940 nm, may exit the optical modulator 70 through waveguides 90 and be emitted into the patient's tissue 34 through emitter 94. The emitted light may then be absorbed and/or scattered through the patient's tissue 34 and may be collected by light collector 96.

In one embodiment, a light collector 96, such as an optical fiber element, may forward the collected light through waveguides 92 back into the optical modulator 70. The optical modulator 70 may then modulate the optical wavelengths and subsequently an active detector 102 may then convert the optical signal into an electrical signal which may then be sent to the amplifier 50 (shown in FIG. 2) and eventually to microprocessor 38. In this embodiment, the modulator 70 may be used to aid the active detector 102 by identifying the light signals of interest and pulling them out of noise bands. Microprocessor 38 may then calculate various spectrophotometric parameters of interest. Microprocessor 38 may also communicate with monitor 12 (shown in FIG. 2) through a wireless module 20 and transmit the various spectrophotometric parameters of interest as well as other data. Though FIG. 3 depicts microprocessor 38 as residing on the optical chip 86, other embodiments may include a microprocessor 38 residing separately from the optical chip 86. Power savings may be achieved by using passive emitters and detectors because they do not require direct electrical power for their operation but instead rely on ambient light present in the environment of the sensor.

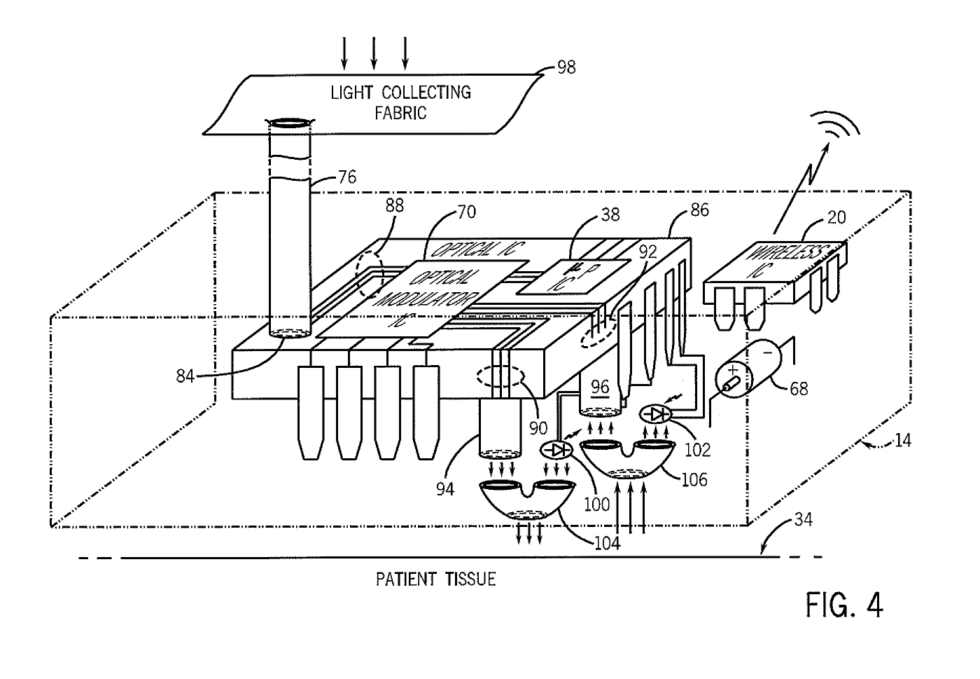

Turning to FIG. 4, the figure depicts an embodiment wherein the ambient light collector may be comprised of a light collecting fabric 98 suitable for collecting and transmitting ambient light incident upon the fabric 98. The light collecting fabric 98 may be formed from woven or knit fiber optic fibers or bundles. The ambient light collecting fabric 98 may be woven into any number of sizes and shapes, including hospital garments (e.g., gowns) or portions of such garments, fabric patches, bandages, and so forth. Larger fabric sizes may result in the collection of more ambient light. Ambient light collected by the fabric 98 may be sent to an optical chip 86 through a waveguide 76. The waveguide 76 may be comprised of fiber optic cable, fiber optic bundles, or any other material that may guide light and which is suitable for connecting the light collecting fabric 98 to the optical chip 86. Ambient light may enter the optical chip 86 through a light-receiving aperture 84, as discussed above with respect to FIG. 3.

The embodiment of FIG. 4 also shows the use of multiple light detectors and/or light emitters. More specifically, the passive emitter 94 and passive light collector 96 discussed above are shown with active emitter 100 and active detector 102. The active emitter 100 may be embodied as an LED, and the active detector 102 may be embodied as a photodiode, for example. It is to be understood that multiple passive emitters 94, active emitters 100, light collectors 96 and/or active detectors 102 may be used in different embodiments. In pulse oximetry applications, for example, two LEDs may be provided as the active emitter 100. In one embodiment, one LED may be configured to emit light at a wavelength near 620-700 nm and a second LED configured to emit light at a wavelength near 860-940 nm, as may be suitable for performing pulse oximetry. The combination of active emitters 100, passive emitters 94, light collectors 96, and/or active detectors 102 may provide operational flexibility in different environments. Specifically, the combination of active emitters 100, passive emitters 94, light collectors 96, and/or active detectors 102 allows for the sensor 14 to continue to operate normally when ambient light conditions are such that the passive emitter 94 and/or light collector 96 may not collect enough light to perform sensor operations using only ambient light, for example, in darkened rooms.

In one embodiment, the light from the passive emitter 94 is combined with the light from the active emitter 100 by using an optic coupler 104. The optic coupler 104 allows for the combining of light from emitters 94 and 100 into a single source of emitted light. This allows for the light to be emitted into a single area of the patient tissue 34. In one embodiment, light may also be collected from the patient tissue 34 and split via an optic splitter 106 so that the collected light enters either the light collector 96 or the active detector 102. The optic splitter 106 allows light collected from a single area of the patient tissue 34 to be processed by different or alternative mechanisms, if desired. Other embodiments may not have an optical splitter 106 and/or an optical coupler 104 or may instead emit or collect light at different areas of the patient tissue 34.

Combining active and passive emitters and/or detectors may reduce energy consumption. For example, a spectrophotometric sensor using LEDs to emit light may drive the LEDs at close to full power in order to generate enough light to penetrate the patient tissue 34. However, techniques disclosed herein may allow for active emitters, if present, to be driven at less than full power by combining the light output of the optical modulator 70 (derived from ambient light) with the light emitted by the active emitters.

In one embodiment, the optical modulator 70 and/or a light meter provided upstream or downstream of the optical modulator 70 may measure the intensity of ambient light that can be collected and/or utilized for transmission into the patient tissue 34. Based on this information, a circuit or other mechanism may be provided that drives the active emitter 100 at a power level needed to supplement the emission profile of the passive emitter 94 so as to maintain a desired or prescribed threshold level of light intensity incident on the tissue 34. That is, if the ambient light collected is sufficient to provide light emission by the passive emitter 94 at a threshold level, the active emitter 100 may not be driven, i.e., is off. If the ambient light collected is insufficient to provide light emission by the passive emitter 94 at the threshold level, the light drive circuitry 48 (shown in FIG. 2) may be directed to drive the active emitters 100 to make up the difference. For example, if the ambient light is only enough for 40% of the required light output, the active emitters may be driven so that they generate the remaining 60% of the required light output. Pulse-width modulation (PWM) may be used to drive the LEDs at less than 100% power but other techniques such as decreasing the forward current of the LEDs may also be used. These techniques allow for savings in power usage because the active emitters are powered only in low light conditions. In well-lit environments, such as hospitals, it may be possible to rely on the ambient light to drive all or a majority of the light output.

With the foregoing in mind, it may be appreciated that an optical modulator 70 as described herein may perform a variety of functions on collected ambient light and/or light transmitted or reflected from a patient tissue 34. In one embodiment, the optical modulator 70 may filter collected ambient light such that only wavelengths of interest are transmitted to the passive emitters 94. For example, FIG. 5 depicts a spectrum 108 showing the intensity of ambient light, in the form of fluorescent light, at different wavelengths. Such fluorescent light may be commonly used for indoor lighting, such as in hospitals and clinics. It should also be understood, however, that the ambient light may be sunlight, incandescent lighting, LED lighting, or other suitable types of lighting that may be used in the vicinity of a patient undergoing monitoring. Indeed, the term “ambient light” as used herein may generally include white light or other broad spectrum light sources having a generally continuous emission spectrum up through and/or including wavelengths in the ultraviolet, near infrared, and infrared spectra.

In one embodiment, optical devices that use fiber Bragg grating (FBG) techniques may provide inline filtering and/or conversion of ambient light, as discussed herein. Such FBG techniques, alone or in combination with other optical techniques (such as Mach-Zehnder modulation) may allow for the creation of filters and converters that may be used in optical chips like chip 86 (shown in FIGS. 3, 4).

Returning now to FIG. 5, in one embodiment the ambient light may be filtered by the optical modulator 70 (shown in FIGS. 2-4) using a 660 nm red FBG bandpass filter and a 900 nm infrared FBG bandpass filter in alternation, i.e., the ambient light may be alternatingly filtered by the two filters such that only one filter is applied at a time. The result of using the two FBG bandpass filters is that two discrete wavelength ranges of light useful in spectrophotometry may be passed through the filters, one at near 660 nm (band 110) and another at near 900 nm (band 112) while the remainder of the filtered spectrum 114 is substantially reduced or eliminated. It is to be noted that any number of discrete wavelength ranges may be produced using the proper bandpass filters. The discrete wavelengths passed through the filter(s) (e.g., 660 nm and 900 nm) may be emitted as described above and used to monitor spectrophotometric parameters of interest. The optical modulator 70 (FIGS. 2-4) may also communicate with other sensor components, such as a microprocessor, in order to communicate the amount of filtered light being emitted as well as to receive instructions related the timing of the emission of filtered light.

It may be noted that any filtering approach that results in the production of a specific, discrete wavelength range at certain locations of the light spectrum may be used. Filtering approaches may utilize Bragg grating filters in general, such as Mach-Zehnder interferometer filters, Michelson interferometer filters, Fabry-Perot filters, moiré filters, and others. Other filter embodiments may utilize dichroic filters, absorptive filters, polarization-rocking filters, nanomechanical filters, and others. It may also be noted that in some embodiments the filtering component may be included as part of the waveguides, for example as part of fiber optic fibers or bundles. In other embodiments the filtering component may be included as part of the ambient light collector, for example by using a dichroic bandpass filter on top of a lens-like structure. That is, a filtering component may be used in any section of the path that ambient light may travel through.

In one embodiment, ambient light may be collected and emitted into the patient tissue 34 without filtering into discrete wavelengths, i.e., the broad spectrum of ambient light may be emitted into the patient tissue 34. The light emerging from the tissue 34 may be collected and transmitted to an optical modulator 70 (FIGS. 2-4). The optical modulator 70 may filter the collected light using embodiments described above such that only wavelengths of interest are processed. For example, the filtering of collected light may result in only certain wavelengths of interest being analyzed by the spectrophotometric system, for example, wavelengths at near 660 nm and at near 900 nm. Various spectrophotometric measures may then be derived by analyzing the collected, filtered light.

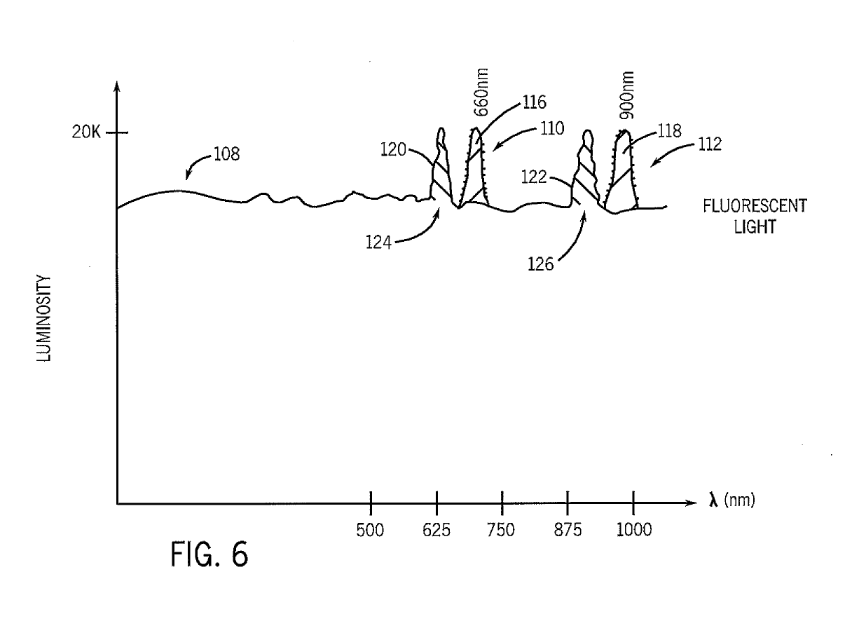

Turning to FIG. 6, a spectrum 108 of the intensity of ambient light in the form of fluorescent light is again depicted. FIG. 6, however, depicts the conversion of light at certain wavelengths to wavelengths of greater interest, such as by use of the optical modulator 70. In one such example, wavelengths of light useful in spectrophotometry, such as those wavelength ranges near 660 nm (band 110) and near 900 nm (band 112), may show an increase (shaded regions 116, 118, respectively) in luminosity after some portion of light at other wavelengths (shaded regions 120, 122, respectively) are optically converted to the wavelengths of interest. The optical modulator 70 may perform wavelength conversion in addition to or instead of wavelength filtering, as discussed above. In one embodiment, the optical modulator 70 may convert the ambient light through frequency shifting, phase shifting and/or amplification. In such an embodiment, the optical modulator 70 may be used to optically shift the frequency and/or phase of certain wavelengths, such as those found in fluorescent lighting close to 660 nm (band 124) and close to 900 nm (band 126), to one or more desired wavelengths.

One reason for choosing the particular wavelength ranges for conversion, such as the bands 124 and 126 found close to 660 nm and 900 nm respectively, may be because the wavelength ranges to be converted may have higher intensities because of the light-emitting composition used in the manufacture of fluorescent tubes. Thus, in one embodiment, the optical modulator 70 may shift a portion of light at wavelength ranges close to 660 nm and 900 nm (shaded regions 120 and 122) to wavelength ranges near 660 nm and near 900 nm (shaded regions 116 and 118). This shifting may result in wavelengths at ranges near 660 nm and near 900 nm having higher luminosity than would otherwise be expected based on the spectrum of the ambient light. Other ambient environments may have different light spectra intensities and wavelengths closer to or further away from those depicted in regions 120 and 122 may be chosen to be shifted.

Several approaches may be used to shift or convert light of one wavelength to another, more useful wavelength. In one embodiment, an on-chip Mach-Zehnder optical modulator may be used, for example, to shift the frequency and/or the phase of the light waves. The Mach-Zehnder modulator may use optical interferometry techniques such as difference frequency generation (DFG), four-wave mixing (FWM), and cross-phase modulation, among others. These techniques may result in the shifting or conversion of wavelengths of light to other, more useful wavelengths. In one embodiment, Mach-Zehnder modulators made of plastic or materials having similar optical properties may be incorporated or etched into an optical chip. Other optical modulators may be used, for example, Michelson modulators, Sagnac modulators, Fabry-Perot modulators, and others. It is to be understood that any embodiment of an optical convertor that is able to shift or convert a wavelength of light to another wavelength of light may be used.

In another embodiment, optical amplification approaches such as the use of a semiconductor optical amplifier (SOA) may be used by the optical modulator 70 (shown in FIGS. 2-4) to amplify light at select wavelengths. For example, a SOA may be used by an optical chip component that amplifies light at certain wavelengths, such as near 660 nm (band 110) and near 900 nm (band 112). The amplification of the light may result in wavelength ranges near 660 nm and near 900 nm having higher luminosity when emitted onto patient tissue than what is observed in the ambient environment. The amplifying of the light may result in the wavelengths of interest reaching a luminosity level that may be useful as part of a spectrophotometric system. Higher luminosities of light at discrete wavelength may also allow reduction in the use of active light emitters, such as LEDs, and may therefore help conserve battery power.

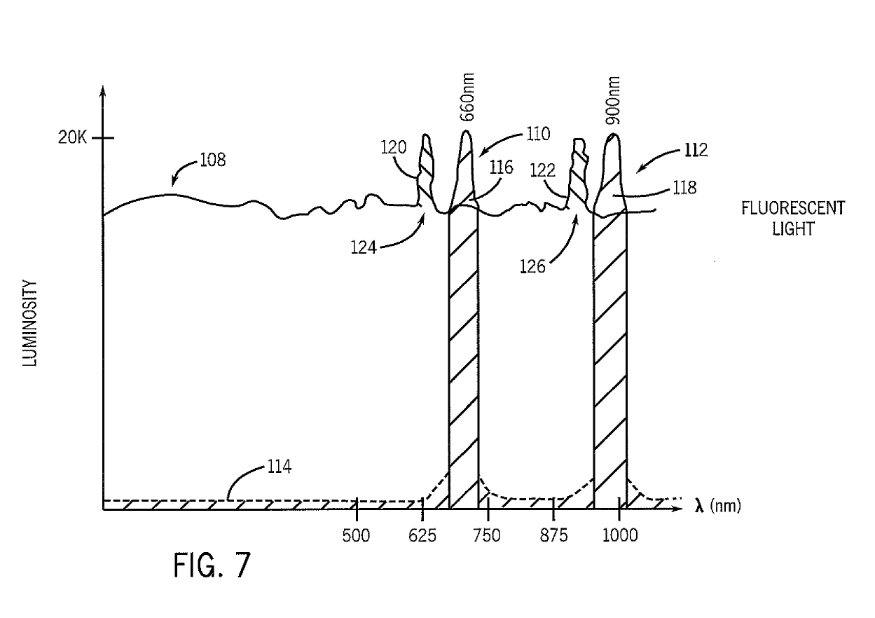

FIG. 7 depicts the results of combining the filtering of light of FIG. 5 with the converting of light of FIG. 6 as discussed above. In the depicted illustration, light at wavelengths close to 660 nm (band 124) and close to 900 nm (band 126) has been shifted, i.e., converted, as discussed above, into light at wavelengths near 660 nm (band 110) and near 900 nm (band 112). The resulting light spectrum may then be filtered, as discussed above, such that light at undesired wavelengths is not transmitted through the filter, yielding a filtered spectrum 114 where the amount of light at wavelengths 660 nm (band 110) and 900 nm (band 112) is increased above what is present in the ambient light due to the conversion of light at other wavelengths. As mentioned above, several filtering and/or converting approaches may be employed, including Bragg grating filters, dichroic filters, absorptive filters, polarization-rocking filters, nanomechanical filters, Mach-Zehnder modulators, Michelson modulators, Sagnac modulators, Fabry-Perot modulators, and others. The filtering of ambient light into wavelengths of interest, for example wavelengths at near 660 nm and 900 nm, may occur after the optical conversion process has increased the luminosity of ambient light to useful levels. The combined conversion and filtering of ambient light may be useful because the resulting light waves may have the desired intensity and wavelength ranges for use in a given spectrophotometric system. Furthermore, less power may be used to the extent that the filtering and conversion process allow the use of ambient light in a monitoring or sensing procedure, thus extending battery life of a wireless sensor.

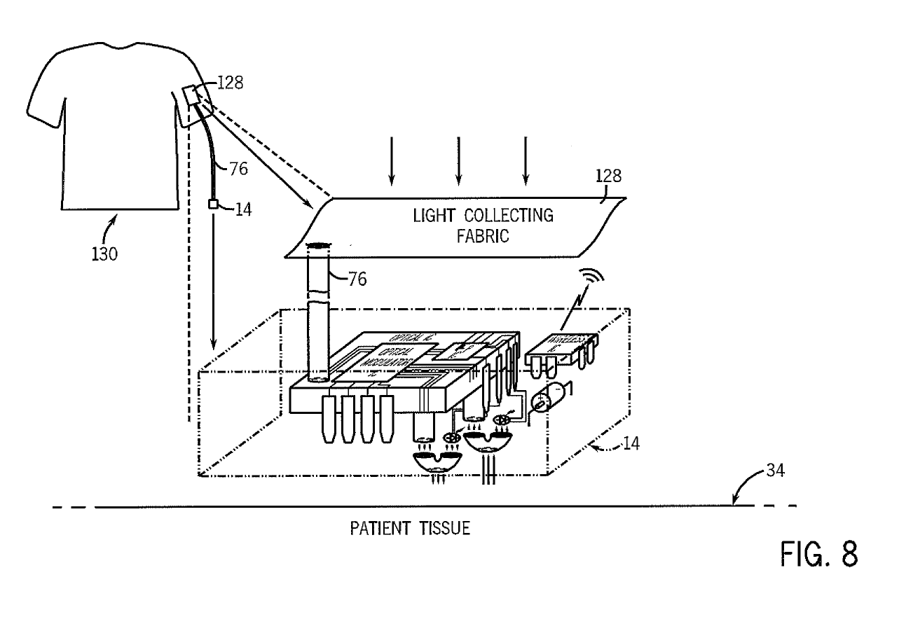

Turning to FIGS. 8, 9, 10 and 11, the depicted embodiments show different configurations of the sensor, ambient light collector, and waveguide attachments discussed herein. For example, FIG. 8 depicts an embodiment wherein an ambient light collecting fabric patch 128 has been incorporated into a garment 130. In one embodiment, the ambient light collecting fabric patch 128 is connected to sensor 14 through a waveguide 76. Sensor 14 may be placed on a patient's tissue 34, such as a finger, toe, earlobe, forehead, and so forth. Sensor 14 may use ambient light to conserve power while monitoring various physical measurements of the patient.

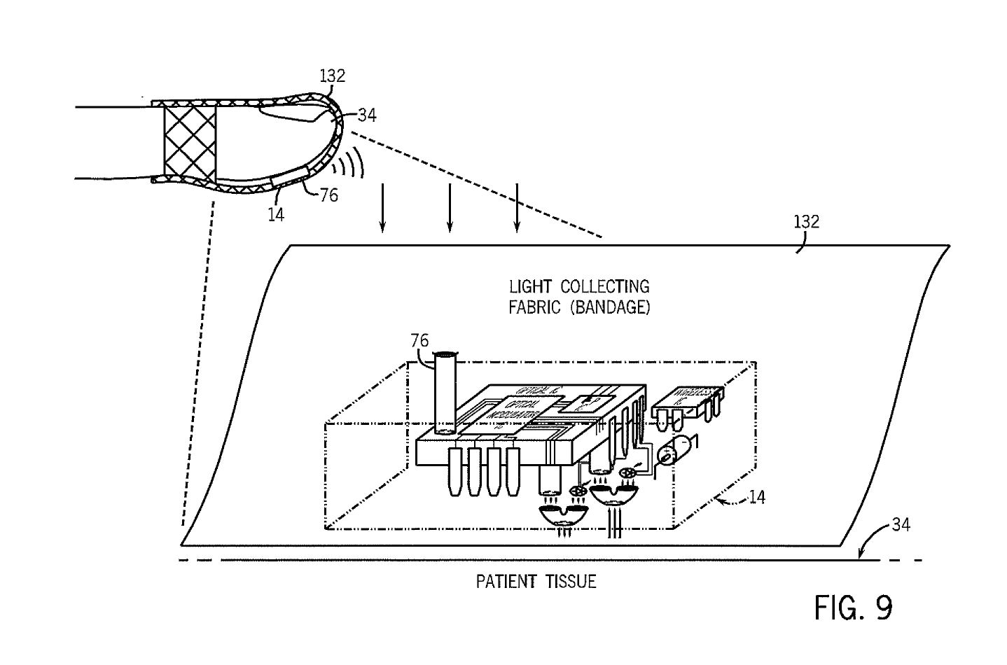

FIG. 9 illustrates an embodiment wherein an ambient light collecting fabric has been woven into a bandage 132 that is used to fasten the sensor 14 to the patient (such as on the finger or forehead). In one embodiment the ambient light collecting bandage 132 may transmit light to sensor 14 through a waveguide 76. Sensor 14 may be placed beneath the bandage 132 and next to the tissue of the patient so that ambient light is collected above the sensor 14 by the structure of the bandage 132 and then transmitted to the underlying sensor 14. In such an embodiment, sensor 14 may then use ambient light to conserve power while monitoring various physical measurements of the patient.

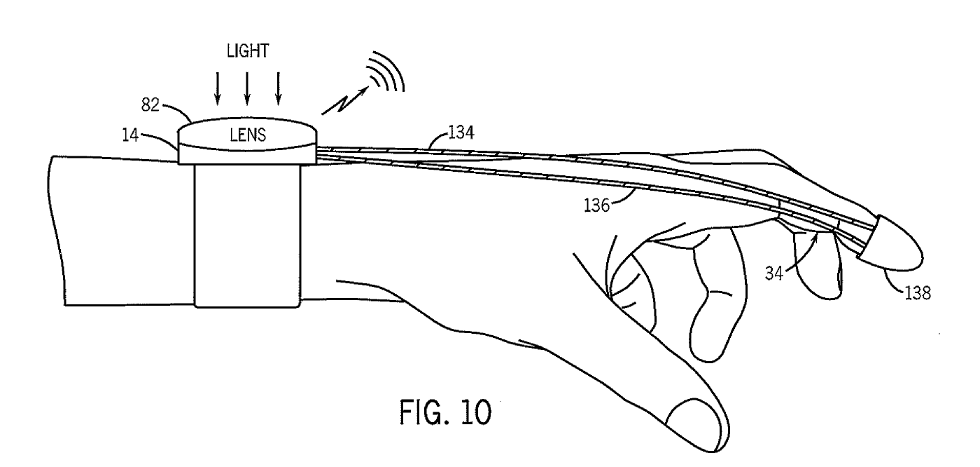

As depicted in FIG. 10, in one embodiment a lens-like structure 82 used to collect the ambient light may be provided as part of the sensor 14 and/or may communicate with the sensor 14 as mentioned previously in reference to FIG. 3. The sensor 14 may be worn by the patient at the measurement site or a proximate location. For example, in one embodiment, the sensor 14 may be provided as a watch-like device worn on the wrist of the patient while the measurement site may be the fingertip of the patient. In such an embodiment, two waveguides 134 and 136 may connect the sensor 14 to a fingertip probe 138 having structures for emitting light into and collect light from the patient tissue 34. For example, waveguide 134 may be used to transmit light from sensor 14 through the fingertip probe 138 into the patient tissue 34. Waveguide 136 may then be used to transmit the light collected from the patient tissue 34 through the finger probe 138 into sensor 14. Thus, in such an embodiment, the waveguides 134, 136 may allow the sensor 14 to be comfortably worn in an area which may be separate from the region on the patient that is best suited for the gathering of spectrophotometric measurements.

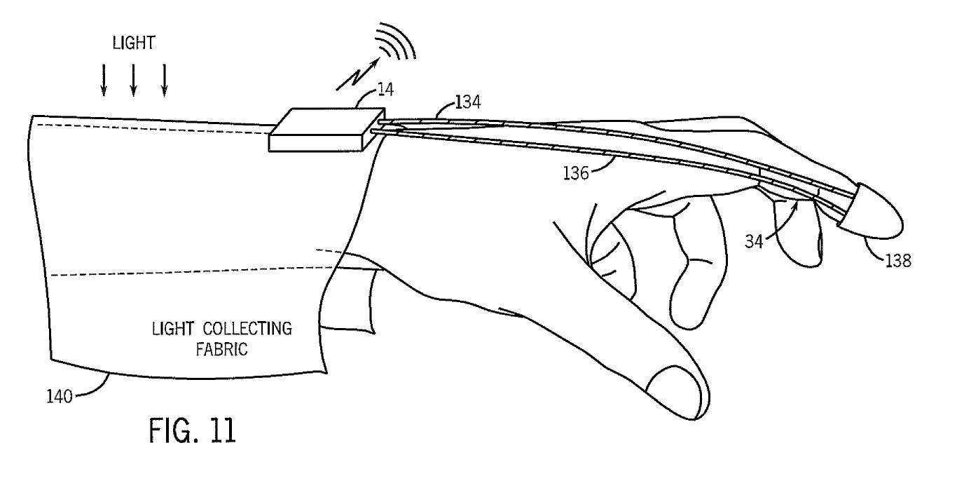

As depicted in FIG. 11, in one embodiment an ambient light collecting fabric may be woven into all or a portion of a light collecting garment 140 so that light may be collected by the garment. In one such embodiment, the sensor 14 may be attached to or communicate with a sleeve of a garment 140 or to some other portion of the garment so that light collected by the garment may be transmitted to the sensor 14 and, from there, to the measurement site. Two waveguides 134 and 136 may join sensor 14 to a fingertip probe 138 as discussed above such that light may be transmitted to and from the fingertip probe 138. The use of the waveguides 134, 136 may allow the sensor 14 to be comfortably worn on or near the garment 140 (such as on the sleeve) which may be separate from the region on the patient 34 that is best suited for the gathering of spectrophotometric measurements.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents or other constituents suitable for spectrophotometric analysis. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. A spectrophotometric sensor, comprising:
an ambient light collecting structure, wherein the ambient light comprises a continuum of wavelengths;
an optical modulator capable of modulating, filtering, or modulating and filtering the ambient light to transmit one or more discrete wavelengths;
a light emitter capable of emitting the one or more discrete wavelengths of light; and
a light detector capable of detecting the emitted light.

2. The spectrophotometric sensor of claim 1, comprising wireless transmission circuitry capable of wirelessly transmitting at least data derived from the light detector.

3. The spectrophotometric sensor of claim 1, wherein the ambient light collecting structure comprises at least one of a lens-like structure or a material of woven fibers.

4. The spectrophotometric sensor of claim 1, wherein the optical modulator comprises at least one of a Mach-Zehnder modulator or a grating filter.

5. The spectrophotometric sensor of claim 1, comprising an electrically powered light emitting device configured to supplement the emitted filtered ambient light.

6. The spectrophotometric sensor of claim 5, wherein the electrically powered light emitting device and the light emitter capable of emitting filtered ambient light are capable of combining their light emissions.

7. A spectrophotometric sensor, comprising:
an ambient light collecting structure, wherein the ambient light comprises a continuum of wavelengths;
an optical modulator capable of converting a first set of one or more discrete wavelengths of ambient light to a second set of one or more discrete wavelengths and of filtering out wavelengths of ambient light other than the second set of one or more discrete wavelengths;
a light emitter capable of emitting the second set of one or more discrete wavelengths; and
a light detector capable of detecting the emitted light.

8. The spectrophotometric sensor of claim 7, comprising wireless transmission circuitry capable of wirelessly transmitting at least data derived from the light detector, wherein the wireless communication protocol used by the wireless transmission circuitry comprises at least one of IEEE 802.15, ZigBee, WirelessHart, MiWi, Bluetooth or IEEE 802.11 protocols.

9. The spectrophotometric sensor of claim 7, wherein the ambient light collecting structure comprises at least one of a lens-like structure or a material of woven fibers.

10. The spectrophotometric sensor of claim 7, wherein the optical modulator comprises at least one of a Mach-Zehnder modulator, Michelson modulator, Sagnac modulator, Fabry-Perot modulator, or a grating filter.

11. The spectrophotometric sensor of claim 7, comprising an electrically powered light emitting device configured to supplement the emitted second set of one or more discrete wavelengths.

12. The spectrophotometric sensor of claim 11, wherein the light emitter emits the one or more discrete wavelengths obtained from the optical modulator in combination with light emitted at the one or more discrete wavelengths by one or more LEDs

13. A monitoring system, comprising:
a monitor, comprising:
a receiver capable of receiving a wireless signal;
a processor capable of processing the wireless signal to generate one or more physiological measurements; and
a display for displaying the one or more physiological measurements;
a wireless sensor, comprising:
an ambient light collector capable of collecting ambient light;
at least optical modulating structure capable of modulating, filtering, or modulating and filtering the ambient light to transmit only one or more discrete wavelengths, converting wavelengths of the ambient light into the one or more discrete wavelengths, or both filtering and converting the ambient light such that only discrete wavelengths are transmitted;
a light emitter capable of emitting the discrete wavelengths of light;
a light detector capable of detecting the emitted lights and generating a corresponding output; and
a transmitter capable of transmitting the wireless signal in response to the output.

14. The monitoring system of claim 13, wherein the sensor comprises an electrically powered light emitting device configured to supplement the emitted discrete wavelengths of light.

15. The monitoring system of claim 14, wherein the wireless sensor comprises a processor capable of processing the corresponding output or a signal derived from the corresponding output to generate the wireless signal.

16. A method of transmitting light into a patient tissue, comprising the acts of:
collecting ambient light comprising a continuum of wavelengths;
transmitting one or more discrete wavelengths of light by modulating, filtering, or modulating and filtering the ambient light to remove all but the one or more discrete wavelengths, by converting other wavelengths of the ambient light to the one or more discrete wavelengths, or by performing one or both of filtering and converting to generate the one or more discrete wavelengths of light from the ambient light; and
directing the one or more discrete wavelengths of light into a patient tissue.

17. The method of claim 16, comprising emitting light from an electrically powered light emitting device in conjunction with the one or more discrete wavelengths of light directed into the patient tissue.

18. The method of claim 16, wherein transmitting the one or more discrete wavelengths comprises transmitting two discrete wavelengths of light in alternation.

19. The method of claim 16, comprising collecting light emerging from the patient tissue and processing the collected light to derive one or more physiological measurements.

20. The method of claim 16, wherein the ambient light is filtered or converted using at least one of a Mach-Zehnder modulator or a grating filter.
